(19) United States
(12) Patent Application Publication
(10) Pub. No.: US 2008/0262427 A1
(43) Pub. Date: Oct. 23, 2008

(54) AUTO-INJECTOR ACTIVATION TRIGGERING ELEMENT

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 12/113,597

(22) Filed: May 1, 2008

Related U.S. Application Data
(63) Continuation of application No. PCT/CH2006/000603, filed on Oct. 31, 2006.

(30) Foreign Application Priority Data
Nov. 3, 2005 (DE) 10 2005 052 502.4

(51) Int. Cl. A61M 5/00

(52) U.S. Cl. 604/131

(57) ABSTRACT

An injection device, e.g. an automatic injector, including a switching element that can be displaced along a longitudinal axis of the injection device from a starting position to a release position via a coupling position, and a triggering element that can be displaced transversely to the longitudinal axis, wherein the switching element includes a control surface element and the triggering element slides along the control surface element at least during the movement of the switching element from the starting position to the coupling position.

### CROSS-REFERENCE TO RELATED APPLICATIONS

This application is a continuation of International Patent Application No. PCT/CH2006/000603 filed Oct. 31, 2006, and claims priority to German Application No. DE 10 2005 052 502.4 filed Nov. 3, 2005, the contents of both of which are incorporated in their entirety herein by reference.

### BACKGROUND

The present invention relates to devices for delivering, dispensing, infusing, administering or injecting a substance, and to methods of making and using such devices. In some embodiments, the present invention relates to an injection device for administering an injectable product, such as a liquid drug such as insulin, heparin, a growth hormone or an osteoporosis preparation. In some embodiments, the injection device can be an injection pen.

German utility model specification 203 19 648 discloses an injection device which comprises a switching element and a triggering element. The switching element can be moved, approximately along a longitudinal axis of the injection apparatus, from an initial position into a release position via a coupling position. The triggering element comprises a guiding surface which, when the switching element is in the coupling position, can be moved in a movement transverse to the longitudinal axis, wherein the guiding surface slides off on a cam of the switching element, such that the switching element is moved in the proximal direction. The user of the device can only start the injection process by pressing the triggering element when the switching element is in the coupling position. The switching element is moved into the coupling position by pressing a needle covering element onto the point of injection. The user of the injection apparatus cannot tell whether the injection apparatus has been placed firmly enough onto the point of injection that the switching element is in the coupling position, whereby it would be possible to inadvertently, accidentally or prematurely trigger the device.

### SUMMARY

An object of the present invention is to provide an injection device in which the user of the device notices when the device is ready for triggering. It is another object of the present invention to provide a method by which such a device can be made and/or operated.

In one embodiment, the present invention comprises an injection device, e.g. an automatic injector, which also might be known or referred to as an auto-injector, including a switching element that can be displaced along a longitudinal axis of the injection device from a starting position to a release position via a coupling position, and a triggering element that can be displaced transversely to the longitudinal axis, wherein the switching element includes a control surface element and the triggering element slides along the control surface element at least during the movement of the switching element from the starting position to the coupling position.

In one embodiment, an injection device in accordance with the present invention comprises a switching element that can be displaced along a longitudinal axis of the injection device from a starting position to a release position via a coupling position, and a triggering element that can be displaced transversely to the longitudinal axis, wherein the switching element comprises a control surface and the triggering element slides along the control surface at least during the movement of the switching element from the starting position to the coupling position. The triggering element comprises a guiding surface inclined towards the longitudinal axis, wherein the guiding surface co-operates with the switching element such that the switching element is moved from the coupling position into the release position when the triggering element is actuated while the switching element is in the coupling position. The guiding surface of the triggering element slides along the control surface.

In some preferred embodiments, an injection device in accordance with the present invention may be an auto-injector. The administering of a drug can comprise an injection sequence in which a needle is injected, or driven or inserted, into a body tissue, and a delivery sequence in which a drug, for example a liquid drug, is delivered. Using the present invention, it is possible to activate the delivery sequence or, in some preferred embodiments, the injection sequence which can be followed by the delivery sequence. The present invention assumes that the injection device comprises a switching element which can be moved, approximately along a longitudinal axis of the injection device, from an initial position into a release position via a coupling position. In some embodiments, the device can comprise a housing which comprises two housing parts which can be detachably or non-detachably connected to each other. A screw lock, bayonet lock, latching lock or other suitable connector may be considered for this purpose. A mechanical sequence controller which controls the injection and/or delivery sequence can, for example, be accommodated in a proximal housing part, and a product container can be accommodated in a distal housing part. “Proximal” is understood to mean the side or end opposite the needle, or the rear, and “distal” is understood to mean the side or end of the injection device, e.g. an elongated injection device, which lies at the needle or toward the front end. The product container, e.g. an ampoule, can be inserted, such that it can be exchanged, into the distal housing part or can be formed directly by the distal housing part.

The distal housing part can mount or carry a transmission member which can be shifted relative to the housing in the proximal direction along the longitudinal axis of the injection device, against the force of an elasticity element, for example a spring. The transmission member can be connected to a needle cover or can itself form a needle cover which can comprise an opening at its distal end for a needle attached to the product container to exit. The transmission member can be coupled to the switching element, for example via co-operating facing surfaces, such that the transmission member slaves the switching element in a movement of the transmission member in the proximal direction. Such a movement is, for example, generated when placing and pressing the transmission member or the needle cover onto the body tissue.

A sequence, e.g. the injection sequence or the delivery sequence, can be started depending on the position of the switching element. The switching element may be held in its initial position by an elasticity element, such as a spring, wherein the switching element can be moved, counter to the spring force, into a coupling position and from the coupling position into a release position. When the switching element is in the initial position, it is not possible to trigger or activate a sequence. When the switching element is in the coupling position, a sequence can be activated by moving the switching element into the release position. When the switching element is in the release position, the sequence is activated.

In some embodiments, the injection device further comprises a triggering element which can be moved transversely relative to the longitudinal axis, e.g. approximately perpendicularly to the longitudinal axis. The triggering element and the switching element can be coupled to each other such that they reciprocally move or shift each other. In a movement of the switching element from the initial position into the coupling position, for example, the switching element can move the triggering element transversely to the longitudinal axis against the force of an elasticity element. The elasticity element may be a suitable spring which presses or urges the triggering element in a direction opposite to the movement direction in which the triggering element is moved when the switching element is moved from the initial position into the coupling position. The triggering element can, for example, move the switching element in the distal direction when the triggering element is actuated while the switching element is in the coupling position. The triggering element may be actuated by a user of the device. When the triggering element is actuated, it is moved in the direction opposite to the direction of the movement which the triggering element performs during the movement from the initial position into the coupling position. In the case of a spring triggering element, for example, the spring can act in its actuation direction, wherein the force of the spring is not sufficient for actuating the actuation element.

The switching element is accordingly actuated in a number of stages, e.g. in two stages. The movement of the switching element from the initial position into the coupling position is caused by moving the transmission member in the proximal direction up to an end stopper. At the end stopper, the switching element is in the coupling position and can no longer be moved further by the transmission member. The second stage of the movement in the distal direction, i.e. from the coupling position into the release position, can only be performed by actuating the triggering element, whereby the movement of the triggering element is converted into a movement of the switching element.

To this end, the switching element can comprise a guiding surface element. The guiding surface element and the switching element can be integral. The guiding surface element can, for example, be a cam-shaped projection extending from the switching element. In this embodiment, during the movement of the switching element from the initial position into the coupling position, the triggering element slides along the guiding surface element, along a guiding surface of the guiding surface element, wherein the elasticity element presses the triggering element against the guiding surface element, against the guiding surface. The guiding surface element can also comprise a number of guiding surfaces, wherein the guiding surface element comprises at least one guiding surface which is inclined towards the longitudinal axis of the device. The at least one guiding surface can exhibit a variable or constant inclination or gradient toward the longitudinal axis. The guiding surface element can comprise a number of constantly or variably inclined guiding surfaces which are joined, wherein their inclinations or gradients can be discontinuous at the transitions at which the guiding surfaces meet. In some preferred embodiments, a guiding surface can also be provided which can be approximately parallel to the longitudinal axis of the device. In some preferred embodiments, at least one guiding surface—e.g. the guiding surface along which the triggering element slides during the movement from the initial position into the coupling position, and is thus moved transverse to the longitudinal axis—points or extends in the proximal direction and transversely to the longitudinal direction, for example toward the triggering element.

In some preferred embodiments, the housing, e.g. the proximal housing part, comprises a housing portion which comprises an opening which enables a user to access an actuation element, which is coupled or integrally connected to the triggering element, when the switching element is in the coupling position. In some embodiments, access to the actuation element by the user can be not immediately possible when the switching element is in the initial position. The actuation element can be a button which is arranged on the triggering element and rises above or drops below the height of the housing part, depending on the switched state of the switching element. If, for example, the actuation element is below or approximately flush with the height of the housing in an initial state, the user of the device can visually or haptically tell that the housing element is protruding out of the housing, when the injection device is ready for administering.

In some embodiments, to move the switching element from the coupling position into the release position, the triggering element can drive the switching element. To this end, the triggering element can comprise a guiding surface which is inclined towards the longitudinal axis of the injection device, wherein the guiding surface co-operates with the switching element such that the switching element is moved from the coupling position into the release position when the triggering element is actuated while the switching element is in the coupling position. The guiding surface can point or extend in the proximal direction and in the actuation direction of the actuation element. The guiding surface of the triggering element can be a gear surface which slides along the guiding surface element when the triggering element is actuated. The gear surface can exhibit a continuous gradient or a gradient which is variable along the longitudinal axis of the device. A number of guiding surfaces can be provided on the triggering element which exhibit a discontinuous or a continuous transition at the points at which they are joined.

The triggering element, which, for example, comprises at least one guiding surface, can be cam-shaped, angled or tapered. In some preferred embodiments, at least one of the at least one guiding surface element and/or at least one of the at least one cam-shaped portion of the triggering element is arranged at a distance from a plane which is spanned by the longitudinal axis of the injection device and the movement direction of the triggering element. To this end, the triggering element can be forked, such that it encompasses the longitudinal axis of the injection device or an element arranged around the longitudinal axis of the injection device, e.g. at least one of a mechanism holder, an advancing element, a driven member and an elasticity element which serves to drive the driven member. In some preferred embodiments, a cam comprising a guiding surface is arranged on each fork element. Each cam can also respectively co-operate with one guiding surface element of the switching element.

For an injection, the injection device together with the transmission member, e.g. the needle cover, can be pressed onto the point of injection, wherein the transmission member is shifted in the proximal direction, but without the needle protruding out of the needle cover, such that the needle cannot yet penetrate into the body tissue, wherein the switching element is in the initial position or the coupling position. When the transmission member is shifted over a maximum path, the switching element is slaved or moved over a path which is smaller than or equal to said maximum path, wherein the actuation element is shifted into a position in which it can be actuated. In this position, i.e. when the switching element is in the coupling position, actuating the triggering element moves the switching element over a path which can be set as an axial distance between the transmission member and the switching element. When the device is not being pressed onto anything, the transmission member and the switching element can be respectively held in a distal position or the initial position by a common spring or by one spring each.

In some embodiments, the injection device can comprise an advancing element which can be biased against the force of an elasticity means, such as a spring. The switching element can block or prevent a release movement of a blocking member which blocks the advancing element from moving in the distal direction and, in the release position, releases the advancing element for moving in the distal direction. From when the switching element is in the initial position until the switching element is in the coupling position, the blocking member is held in engagement with the advancing element by the switching element. To this end, the switching element can comprise a holding stage which can be moved along the blocking member along the longitudinal axis, wherein the holding stage can be approximately axially parallel. The blocking member can be formed integrally on a mechanism holder. In some preferred embodiments, the holding stage exhibits at least an axial length which corresponds to the path over which the switching element is moved from the initial position into the coupling position. Thus, it is ensured that between the initial position and the coupling position, the holding stage—which can be integrally formed by the switching element—is approximately at the same axial height as the blocking member, whereby the blocking member is held in engagement with a blocking groove of the advancing element. The holding stage can be followed along the longitudinal axis by a release stage which, during the movement of the switching element from the coupling position into the release position, is shifted at approximately the same axial height or length as the blocking member, such that the blocking member latches or never out of the engagement with the advancing element, whereby an injection sequence can be started. In some preferred embodiments, the release stage follows the holding stage on a side opposite to the movement direction of the switching element from the coupling position into the release position. In some preferred embodiments, the holding stage exhibits a smaller distance from the longitudinal axis than the release stage.

The released advancing element is shifted in the distal direction until it latches via a blocking element into the mechanism holder, whereby a driven member is released, such that the driven member can be moved relative to the advancing element in the distal direction for a product delivery.

In some embodiments, the present invention also relates to a method for triggering a triggering mechanism of an injection device. Firstly, a triggering element can be pressed onto a guiding surface of a switching element. The switching element is then moved from an initial position, along a longitudinal axis of the injection device, into a coupling position, wherein the pressed triggering element slides along the guiding surface and performs a movement transverse to the movement direction of the switching element. In some preferred embodiments, an effective triggering movement of the triggering element can only then be performed. The movement of the triggering element can be performed transversely to the movement direction of the switching element, whereby the switching element is driven in the same direction as the movement which it performs during the movement from the initial position into the coupling position. The switching element is driven in this same direction until it reaches the release position, whereby the injection sequence and then the delivery sequence are activated.

### BRIEF DESCRIPTION OF THE DRAWINGS

FIG. 1 is a sectional representation along a longitudinal axis of one embodiment of an injection device in accordance with the present invention;

FIG. 2 is a sectional representation along a longitudinal axis of one embodiment of an injection device in accordance with the present invention;

FIG. 3 is a sectional representation along the longitudinal axis, perpendicular to the sectional representation of FIG. 2;

FIG. 4 is a perspective view of one embodiment of a switching element and a mechanism holder in accordance with the present invention;

FIG. 5 is a sectional view of a part of one embodiment of an injection device, in an initial state in accordance with the present invention;

FIG. 6 is a sectional view of the part of the injection device of FIG. 5, after a product delivery;

FIG. 7 is a sectional view of an embodiment of a switching element and a triggering element, in an initial position;

FIG. 8 is a sectional view of the switching element and the triggering element of FIG. 7, in a coupling position;

FIG. 9 is a sectional view of the switching element and the triggering element of FIGS. 7 and 8, in a release position; and

FIG. 10 is an exploded representation, including embodiments of an advancing element, a latching element and an elasticity means.

### DETAILED DESCRIPTION

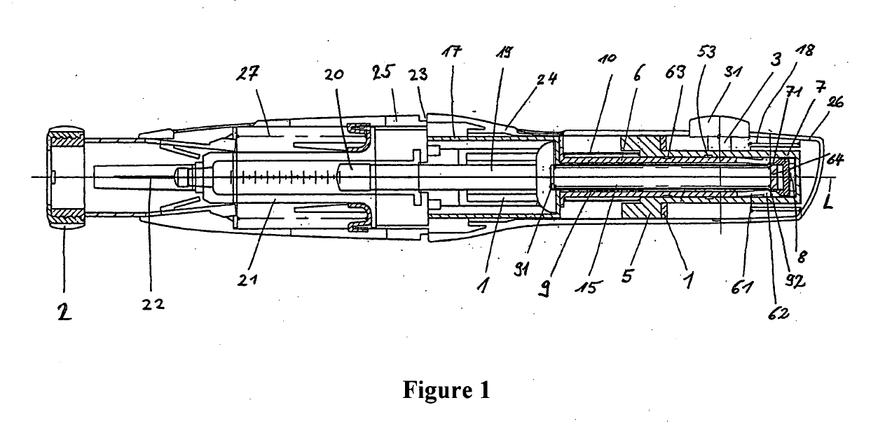

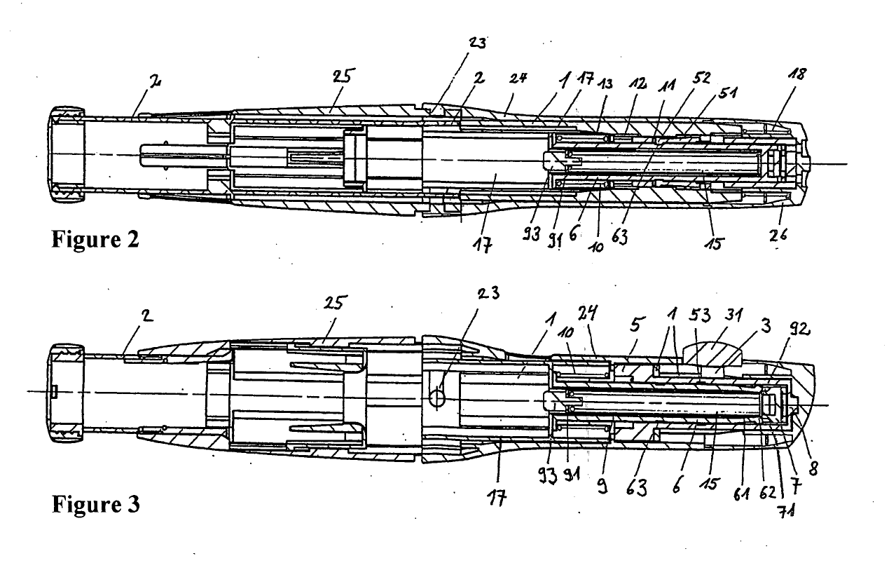

FIG. 1 shows an injection device in the form of an auto-injector. The injection device comprises a mechanism including a mechanical sequence controller, using which a product container 21 contained in the injection device is firstly shifted in the distal direction during an injection sequence, such that the needle 22 fixed to the product container 21 is injected into a body tissue of a patient, wherein after the injection sequence, the piston 20 in the product container 21 is moved in the distal direction during a delivery sequence, such that the product contained in the container 21 is delivered through the needle 22 into the body or body tissue of a patient. Unless specified otherwise, the description with respect to FIG. 1 also relates to FIGS. 2 and 3, in which a similar injection device is shown, without a product container 21.

The injection device comprises a distal housing part 25 and a proximal housing part 24 which are connected by a detachable connection, e.g. a bayonet lock 23. A mechanism holder 5 is accommodated in the proximal housing part 24 and connected, rotationally and axially fixed, to the proximal housing part 24. To axially fix the mechanism holder 5, it is axially enclosed via its facing sides between a protrusion of the housing 24 which protrudes radially inwards and a cap 26 which is proximally fitted onto the housing 24. The mechanism holder 5 encompasses an advancing element 6 which can be shifted relative to the mechanism holder 5 along the longitudinal axis L of the injection device. The outer circumferential surface of the sleeve-shaped advancing element 6 can slide along the inner circumferential surface of the approximately sleeve-shaped mechanism holder 5. A spring 10, e.g. a helical spring 10, is arranged between a distal or forward facing side of the mechanism holder 5 and a proximal or rearward facing side of an annular flange arranged on the distal end of the advancing element 6, and when pressure-biased, said spring charges the advancing element 6 with an advancing force in the distal direction relative to the mechanism holder 5.

The distal facing surface of the annular flange which is distally attached to the advancing element 6 can act, in a direction along the longitudinal axis L, on a cage 17 which can be shifted in the housing along the longitudinal axis L. The cage 17 is coupled to the product container 21 such that when the cage 17 moves in the distal direction, the product container 21 is likewise moved in the distal direction, i.e. in the injecting direction of the needle 22. The movement of the cage 17 in the distal direction cannot yet cause a movement of the piston 20 within the product container 21. The product container 21 and the cage 17 are pressed in the proximal direction of the injection device by a pressure-biased spring 27. The force of the spring 27 with which the cage 17 is pressed in the proximal direction is smaller than the force of the spring 10 with which the advancing element 6 and the cage 17 can be moved in the distal direction when the advancing element 6 is released for moving along the longitudinal axis L, such that the product container 21 can be moved together with the needle 22 in the distal direction, to inject the needle 22 into the body tissue.

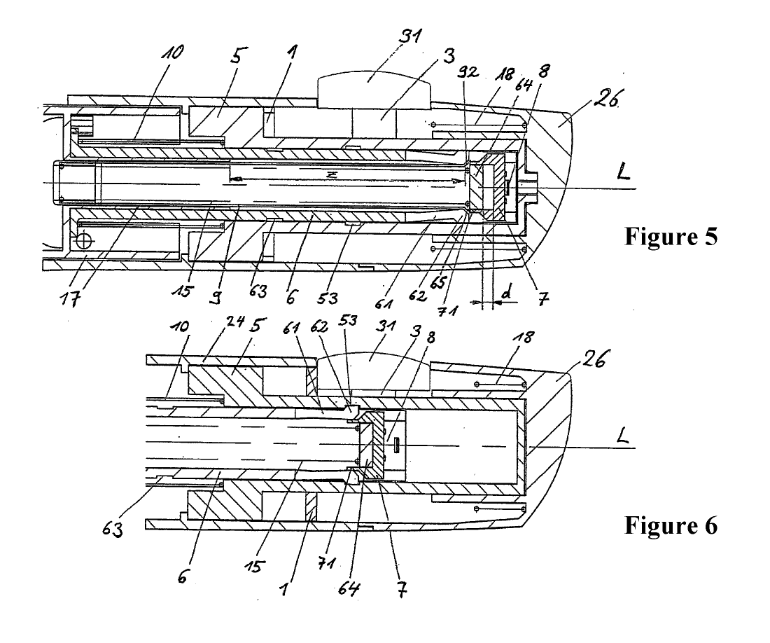

A sleeve-shaped driven member 9 is arranged within the advancing element 6 and can be moved relative to the advancing element 6 along the longitudinal axis L for a delivery sequence, when it is released for axial movement. A collar 92 which protrudes radially outward is situated on the proximal end of the driven member 9. An annular groove is situated between the driven member 9 and the advancing element 6 and has a groove width which is greater than the extent to which the collar 92 projects radially from the driven member 9. To form the annular groove and to mount or carry the driven member 9, a sleeve-shaped portion of the cage 17 engages from the distal side with the inner diameter of the advancing element 9, as can best be seen in FIG. 5.

The distal end of the driven member 9 comprises a facing surface 91 which acts as a stopper surface on which a spring 15, e.g. a helical spring, can be supported. The other end of the spring 15, which can be pressure-biased, is supported on a distal surface of a stopper element 64 which is formed on a proximal area of the advancing element 6. The spring 15, which can exhibit a greater spring constant than the spring 10, can drive the released driven member 9 in the distal direction along the longitudinal axis L. The distal facing side of the driven member 9 or a contact element 93 fixed to it, as shown in FIGS. 2 and 3, come into contact with a piston rod 19 or a domed element arranged on the proximal end of the piston rod 19, whereby the piston 20, which is distally arranged on the piston rod 19, is moved in the product container, whereby the product is delivered through the needle 22 when the driven member 9 is moved in the distal direction.

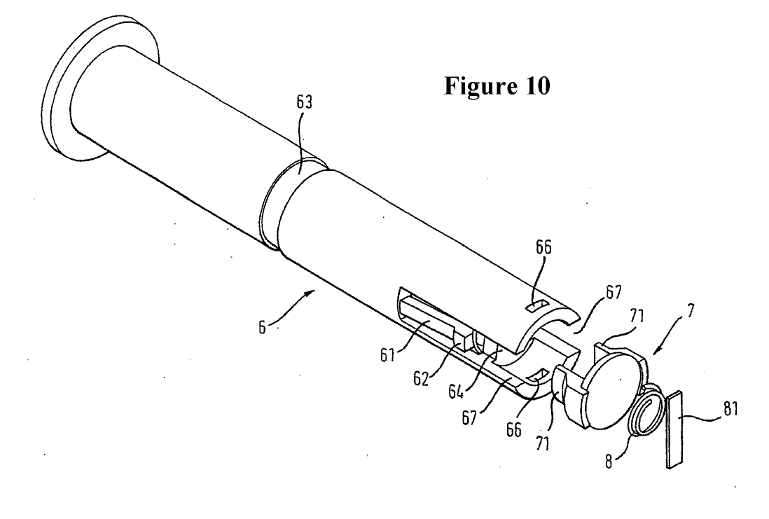

As can best be seen in FIG. 5, a latching element 7 which can be moved relative to the advancing element 6 along the longitudinal axis L is arranged on the proximal end of the advancing element 6. As can be seen in FIG. 10, the latching element 7 is forked and encompasses the longitudinal axis L via two latching portions or arms 71. The latching portions 71 are each guided in a groove-shaped guide 67 (FIG. 10) formed by the advancing element 6. The latching element 7 is also guided by a stopper element 64 which is formed by the advancing element 6 and accommodated between the latching portions 71. As shown for example in FIG. 5, a distance d can exist between the proximal facing side of the stopper element 64 and a distal facing side of the latching element 7, over which the latching element 7 can be moved in the distal direction. As can be seen in FIG. 10, the latching element 7 is charged with a force in the distal direction by a spring 8. The spring 8 is supported on the proximal end of the latching element 7 and on an element which is arranged proximally with respect to it and fixed to the advancing element, i.e. on the holding stay 81. The holding stay 81 can be inserted into the advancing element 6, transverse to the longitudinal axis L, through openings which form a holding stay receptacle 66, and fixed to the advancing element 6 in order to serve as an abutment for the spring 8. The spring 8 can, for example, be a helical spring or a combined helical and spiral spring or a leaf spring. The holding stay 81 can, for example, be formed from a spring material and can exhibit a shape which can charge the latching element 7 directly with a spring force, such that the spring 8 can be omitted, since the latching element 7 assumes the function of the spring 8.

In the position shown in FIG. 5, a movement of the latching element 7 in the distal direction is prevented by the latching element 7 being blocked, via its latching portions 71, each by a gear surface 65 of a blocking element 62. The cam-shaped blocking element 62 is arranged on an arm 61 and is integrally connected, elastically, to the advancing element 6. The gear surface 65 simultaneously serves to block the longitudinal movement of the driven member 9, in the position shown in FIG. 5. To this end, the collar 92 of the driven member 9 abuts the gear surface 65. The latching element 7 can either abut the gear surface 65 directly or abut a proximal portion of the collar 92. If the released advancing element 6 is moved in the distal direction, the blocking element 62 arrives at approximately the same axial height as a latching groove 53, after a certain path length which approximately corresponds to the path over which the product container 21 is shifted for injecting. The latching groove 53 is formed by the advancing element 6. The latching groove 53 can be a recess or an annular circumferential recess for each of the blocking elements 62. As soon as the latching groove 53 and the blocking element 62 are at the same axial height, as shown in FIG. 6, the spring force of the arms 61 and/or the spring force of the spring 8 and/or the spring force of the spring 15 in conjunction with the collar 92 and/or the latching element 7 sliding off on the gear surface 65 of the blocking element 62 causes a movement radially outward, into the latching groove 53. This starts the delivery sequence.

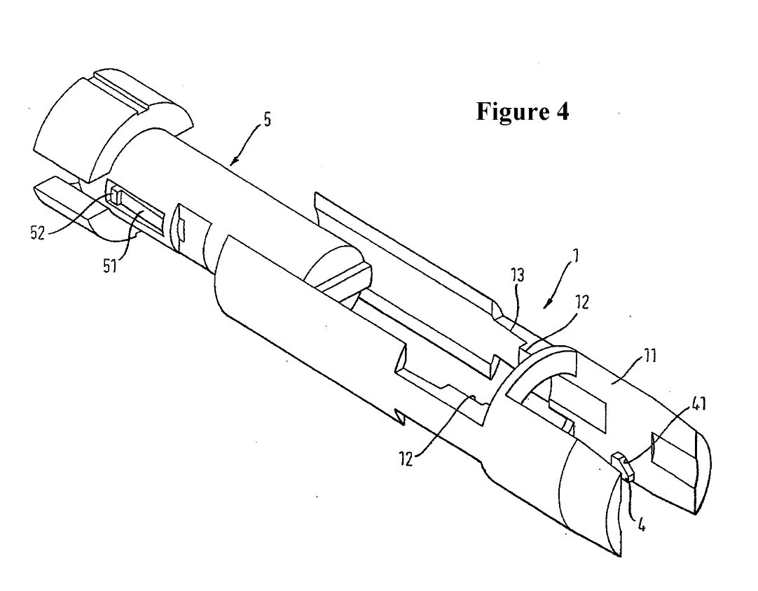

The collar 92 is then no longer blocked by the gear surface 65, whereby the spring 15 drives the driven member 9 in the distal direction and thus drives the piston 20 for a product delivery. The latching element 7 is simultaneously released for axial movement, such that the spring 8 shifts the latching element 7 over the path d (FIG. 5), whereby the latching portions 71 are shifted in front of the blocking element 62. The blocking element 62 is prevented from moving out of the latching groove 53, since the latching portion 71 blocks the movement which the blocking element 62 would perform if it moved out of the latching groove 53. The movement of the driven member 9 can in particular be limited to a path z (FIG. 4), wherein the sleeve-shaped portion of the cage 17 forms the end stopper, or limited to a path which the piston 20 can travel in the product container 21.

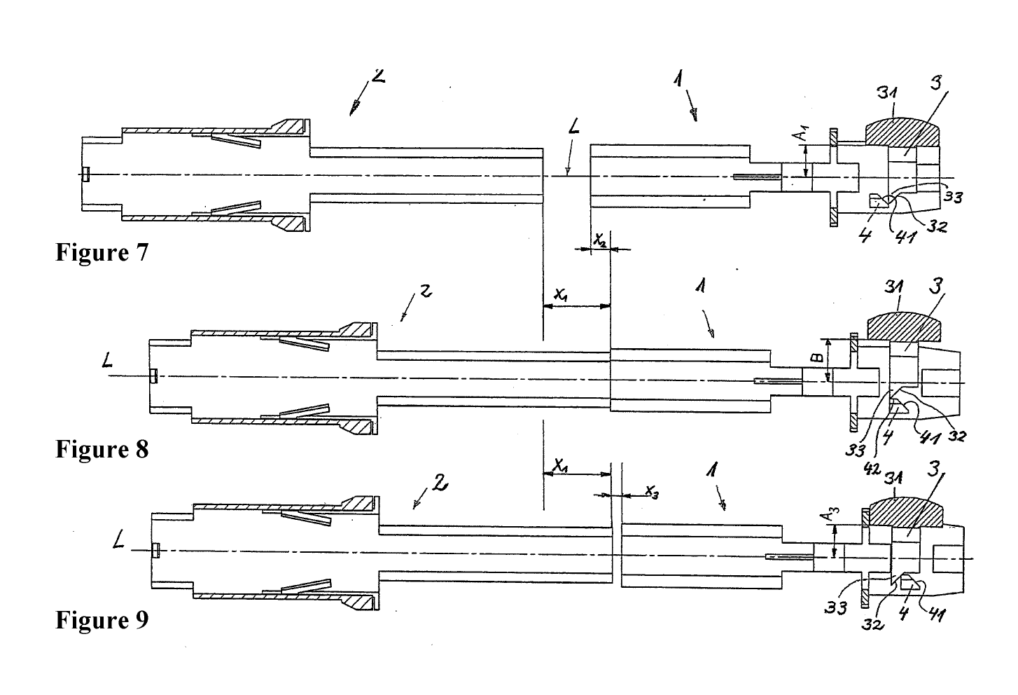

In some preferred embodiments, the delivery movement of the driven member 9 can only be performed once the needle 22 has completely or substantially completely performed its injection movement. In some embodiments, the injection sequence can also only be started once a transmission member 2, formed as a needle cover, has been pressed sufficiently firmly onto the body tissue at the point of injection. When the device is pressed onto the body tissue, the transmission member 2 is shifted relative to the distal housing part 25. The proximal housing part 24 mounts a switching element 1 such that it can be moved along the longitudinal axis L. As shown in FIGS. 7 to 9, the switching element 1 is slaved or moved by the movement of the transmission member 2 in the proximal direction, wherein a proximal facing surface of the transmission member 2 and a distal facing surface of the switching element 1 come into contact. The transmission member 2 is moved in the proximal direction, counter to the force of a spring. The switching element 1 is pressed in the distal direction by a spring 18. The switching element 1 can be moved in the proximal direction, against the force of the spring 18.

The switching element 1 comprises an annular portion which co-operates with the mechanism holder 5 as an axial stopper for a movement of the switching element 1 in the distal direction, when the switching element 1 is in its initial position. The switching element 1 comprises a guiding surface element 4 which comprises a guiding surface 41 which is inclined towards the longitudinal axis L. As shown in particular in FIG. 4, the switching element 1 is forked. A guiding surface element 4 is situated on each of the two fork elements. A triggering element 3, which is likewise forked, comprises a cam 33 on each of its fork elements, which can co-operate with the respective guiding surface element 4. The triggering element 3 can be moved transversely—as shown here—perpendicular, to the longitudinal axis L. The cams 33 and the guiding surface elements 4 are each arranged at a distance from a plane which is spanned by the longitudinal axis L and the movement direction of the triggering element 3. The fork elements of the switching element 1 and the fork elements of the triggering element 3 encompass the longitudinal axis L.

FIG. 7 shows an initial position of the switching element 1. The triggering element 3 is pressed against the guiding surface 41 of the guiding surface element 4 via the cam 33 by an elasticity element, e.g. a spring (not shown), wherein an actuation element 31 connected to the triggering element 3 assumes a position below or flush with the height of the housing 24, 25, as shown in FIG. 6, wherein the position shown in FIG. 6 is an example. When the switching element 1 is in the position shown in FIG. 7, the actuation element 31 cannot or not immediately be actuated, for example by a finger of the user. Actuating the actuation element 31 when the switching element 1 is in the initial position shown in FIG. 7 or in an intermediate position between the initial position and the coupling position does not lead to the sequence controller for the injection being triggered.

When the user of the device presses the transmission member 2 onto the point of injection, it is shifted over the path x₁, whereby the switching element 1 is slaved or moved over the path x₂. During this movement, the guiding surface 41 which is inclined toward the longitudinal axis L slides along the cam 33, whereby the triggering element 3 is firstly shifted transversely to the longitudinal axis L, such that the actuation element 31 rises above the height of the housing 25, whereby the user can access the actuation element 31 or derive an optical indication that the device is ready for triggering the sequence controller, such that the triggering element 3 may be activated. When the switching element 1 is moved further in the proximal direction, a guiding surface 42 travels along the cam 33, wherein the guiding surface 42 runs or extends approximately parallel to the longitudinal axis L, as shown in this example, such that when the guiding surface 42 moves along the cam 33, the triggering element 3 remains stationary. Once the switching element has reached the coupling position shown in FIG. 8, the triggering element 3 or actuation element 31, respectively, can be actuated to effectively trigger the sequence controller.

When, as shown in FIG. 9, the actuation element 31 is pressed transversely to the longitudinal axis L, the guiding surface 32 of the triggering element 3 then slides off on the guiding surface element 4, whereby the switching element 1 is moved over a path x₃ in the proximal direction, such that it assumes a release position. In the release position, the sequence controller of the injection device is started.

As shown in FIGS. 2 and 4, the mechanism holder 5 comprises a blocking member 52 which is elastically arranged via an arm 51 and engages with a latching groove 63 situated on the outer circumference of the advancing element 6. As long as the blocking member 52 engages with the latching groove 63, a movement of the advancing element 6 relative to the mechanism holder 5 is blocked or prevented. When the switching element 1 is in the initial position and in the coupling position, a holding stage 11 formed by the switching element 1 is at the same axial height as the blocking member 52, such that the blocking member 52 is held in engagement with the advancing element 6 by the holding stage 11. When the switching element is moved from the coupling position into the release position, a release stage 12 formed by the switching element 1 is moved to the same axial height as the blocking member 52, whereby the blocking member 52 is pressed out of engagement with the blocking groove 63 due to the elastic arm 51 and/or due to the gear action of the advancing element 6, which is charged with an axial force of the spring 10, and the geometry or shape of the blocking member 52. The distance between the holding stage 11 and the longitudinal axis L is smaller than the distance between the release stage 12 and the longitudinal axis L. Mounting ramps 13 are arranged on the switching element 1 in the extension of the release stage 12 in the distal direction, on which the blocking members 52 are guided onto the holding stages 12 when the switching element 1 is mounted to the mechanism holder 5.

When the blocking members 52 are extended, the advancing element 6 is pressed in the distal direction by the spring 10 until the blocking elements 62 engage with the blocking groove 53, wherein the needle 22 is injected. Once the blocking elements 62 have engaged with the latching groove 53, the delivery sequence described above follows.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An injection device comprising:
a switching element that can be displaced along a longitudinal axis of the injection device from a starting position to a release position via a coupling position; and
a triggering element that can be displaced transversely to the longitudinal axis, wherein
the switching element comprises a control surface and the triggering element slides along the control surface at least during the movement of the switching element from the starting position to the coupling position.

2. The injection device according to claim 1, wherein the triggering element comprises a guiding surface inclined towards the longitudinal axis, wherein the guiding surface co-operates with the switching element such that the switching element is moved from the coupling position into the release position when the triggering element is actuated while the switching element is in the coupling position.

3. The injection device according to claim 2, wherein the guiding surface of the triggering element slides along the control surface.

4. An automatic injection device, comprising:
a) a switching element which can be moved, approximately along a longitudinal axis of the injection apparatus, from an initial position into a release position via a coupling position;
b) a triggering element which can be moved transversely to the longitudinal axis;
c) wherein the switching element comprises a guiding surface element; and
d) the triggering element slides along the guiding surface element, at least when the switching element moves from the initial position into the coupling position.

5. The injection device according to claim 4, further comprising an elasticity element which presses the triggering element against the guiding surface element.

6. The injection device according to claim 4, wherein the guiding surface element comprises at least one guiding surface which is inclined towards the longitudinal axis, whereby the triggering element is moved transversely to the longitudinal axis when the switching element is moved from the initial position into the coupling position.

7. The injection device according to claim 4, further comprising a housing portion which comprises an opening which enables a user to access an actuation element, which is coupled to the triggering element when the switching element is in the coupling position and does not immediately enable said access when the switching element is in the initial position.

8. The injection device according to claim 7, wherein the actuation element comprises a surface which points radially away from the longitudinal axis of the injection device and protrudes beyond an outer surface of the housing portion when the switching element is in the coupling position.

9. The injection device according to claim 7, wherein the actuation element is approximately flush with the outer surface of the housing portion or is below the outer surface of the housing portion when the switching element is in the initial position.

10. The injection device according to claim 4, wherein the triggering element comprises a guiding surface inclined towards the longitudinal axis, wherein the guiding surface co-operates with the switching element such that the switching element is moved from the coupling position into the release position when the triggering element is actuated while the switching element is in the coupling position.

11. The injection device according to claim 10, wherein the guiding surface of the triggering element slides along the guiding surface element.

12. The injection device according to claim 4, wherein the triggering element comprises at least one cam which comprises a guiding surface, and the switching element comprises at least one guiding surface element, wherein at least one of the at least one cam and/or at least one of the at least one guiding surface element is arranged at a distance from a plane which is spanned by the longitudinal axis of the injection device and the movement direction of the triggering element.

13. The injection device according to claim 4, wherein the triggering element is forked to encompasses the longitudinal axis.

14. The injection device according to claim 4, further comprising a transmission member which can be pressed onto the point of injection of a needle without the needle being able to penetrate into the point of injection when the switching element is in the initial position or the coupling position.

15. The injection device according to claim 14, further comprising a needle protection sleeve coupled to the transmission member, which can be shifted over a maximum path (x₁) along the longitudinal axis of the injection device toward the needle against the force of a spring, wherein the switching element is slaved over a path (x₂) which is smaller than or equal to the maximum path (x₁).

16. The injection device according to claim 15, wherein when the switching element is moved over the path (x₂) which is smaller than or equal to the maximum path (x₁), an actuation element is shifted into a position in which it can be actuated.

17. The injection device according to claim 16, wherein actuating the triggering element causes the switching element to be moved over a path (x₃) from the coupling position into the release position, wherein in the release position, a distance exists between the transmission member and the switching element which corresponds to the path (x₃) which the switching element travels when it is moved from the coupling position into the release position.

18. The injection device according to claim 4, further comprising an advancing element which can be biased against the force of a spring, wherein the switching element blocks the advancing element from moving in the distal direction and, in the release position, releases the advancing element for moving in the distal direction.

19. The injection device according to claim 18, wherein in the initial position and in the coupling position, the switching element holds a blocking member in engagement with the advancing element.

20. The injection device according to claim 19, wherein when the advancing element is released a product container associated with the injection device and carrying a needle is shifted along the longitudinal axis by the spring such that the needle is injected into a tissue.

21. The injection device according to claim 20, wherein the advancing element is shifted it latches into a mechanism holder such that it is fixed against shifting, and a driven member is released and relative to the advancing element.

22. A method for triggering a triggering mechanism of an injection device, comprising the steps of:
a) pressing a triggering element onto a guiding surface of a switching element;
b) moving the switching element from an initial position, along a longitudinal axis, into a coupling position, wherein the pressed triggering element slides along the guiding surface and performs a movement transverse to the movement direction of the switching element;
c) moving the triggering element transverse to the movement direction of the switching element, whereby the switching element is driven in the same direction as the movement which it performs during the movement from the initial position into the coupling position.
